(19) United States
(12) Patent Application Publication
(10) Pub. No.: US 2008/0300624 A1
(43) Pub. Date: Dec. 4, 2008

(54) Tissue Stabilizer and Fastener

(75) Inventors: Richard F. Schwemberger, Cincinnati, OH (US); John P. Measamer, Cincinnati, OH (US); Michael S. Cropper, Edgewood, KY (US); Richard C. Smith, Milford, OH (US)

Correspondence Address: WELSH & FLAXMAN LLC, 2000 DUKE STREET, SUITE 100, ALEXANDRIA, VA, 22314, US

(73) Assignee: Ethicon Endo-Surgery, Inc., Cincinnati, OH (US)

(21) Appl. No.: 11/755,475

(22) Filed: May 30, 2007

(51) Int. Cl. A61B 17/03

(52) U.S. Cl. 606/213

(57) ABSTRACT

A surgical instrument for applying a fastener to tissue of a patient. The instrument includes an elongate shaft having a working end and a grip end opposite the working end. The instrument includes opposing stationary jaws mounted on the working end of the elongate shaft defining a gap between the jaws. The gap is sized and shaped for receiving tissue. At least one of the jaws includes a fastener ejector directed toward the gap for introducing a fastener into tissue received in the gap to selectively fasten the tissue with the fastener. The instrument includes a mechanism operatively connected to the fastener ejector for introducing the fastener into the tissue thereby applying the fastener to the tissue of the patient.

### BACKGROUND

This invention generally relates to a surgical instrument, and more particularly to a device for stabilizing and fastening tissue

Gastroesophageal reflux disease or persistent heartburn is caused by an improper relaxation of the lower esophageal sphincter, allowing acidic stomach contents to travel into the esophagus. If left untreated, chronic reflux may cause esophageal stricture, bleeding ulcers, perforation, and scarring. Continued reflux may lead to Barrett's esophagus, involving changes in the esophageal cells and possibly leading to cancer. Antacids and proton pump inhibitors are initially used to treat this condition. If these treatments are unsuccessful, surgical intervention is often recommended.

One interventional surgical method is known as Nissen fundoplication. This procedure involves wrapping a fundus of the stomach around the lower end of the esophagus and fastening it in place to make the lower esophageal sphincter less compliant. Traditionally, this procedure was accomplished by open surgery using sutures to secure the plicated fundus of the stomach around the esophagus without penetrating the stomach. More recently, laparoscopic Nissen procedures have been used. In some laparoscopic procedures, surgical fasteners are used with an endoscopic applicator. Several different fastener designs have been developed. Some of these designs include a two piece fastener. A first of these pieces, a male component, includes a base having two straight elongate needles extending perpendicularly outward from the base generally parallel to each other. A second piece, a female component, includes a receiver element having openings positioned for receiving the needles of the first piece and a lock for holding the needles in place once received in the openings. In use, tissue is gathered, the needles of the first piece are pushed through the gathered tissue and the openings of the second piece to hold the tissue and fastener in place.

Various applicators are used to apply the fastener to the tissue. One applicator includes an elongate shaft having two jaws pivotally attached to its end. The jaws include receptacles for holding the first and second pieces of the fastener. The jaws push the needles of the first piece through the gathered tissue and the openings of the second piece to hold the tissue and fastener in place. As will be appreciated by those skilled in the art, because both jaws are pivotally attached to the shaft, both pieces of the fastener sweep through arcs as the jaws pivot to pierce the tissue and push the needles into the corresponding openings. As a result of the fastener pieces sweeping through arcs, both fastener pieces move relative to the tissue, making precise placement of the fastener in the tissue difficult. Even when the needles engage the tissue, the folded tissue can move relative to the female fastener component before the fastener components are fastened together. Thus, there is a need for an applicator that reduces the opportunity for the tissue to move as the fastener is applied.

### BRIEF SUMMARY

The present invention relates to a surgical instrument for applying a fastener to tissue of a patient. The instrument comprises an elongate shaft having a working end and a grip end opposite the working end. The instrument also includes opposing stationary jaws mounted on the working end of the elongate shaft defining a gap therebetween. The gap is sized and shaped for receiving tissue therein. At least one of the jaws includes a fastener ejector directed toward the gap for introducing a fastener into tissue received in the gap to selectively fasten the tissue with the fastener. Further, the instrument comprises a mechanism operatively connected to the fastener ejector for introducing the fastener into the tissue thereby applying the fastener to the tissue of the patient.

In another aspect, the present invention relates to a surgical instrument for applying a fastener to tissue of a patient. The instrument comprises an elongate shaft having a working end and a grip end opposite the working end. The instrument includes opposing jaws mounted on the working end of the elongate shaft defining a gap therebetween. The gap is sized and shaped for receiving tissue therein. At least one of the jaws includes an channel ending in an opening adjacent the gap for introducing an elongate fastener into tissue received in the gap to selectively fasten the tissue with the fastener. In addition, the instrument comprises a mechanism operatively connected to the channel for introducing the fastener into the tissue longitudinally with respect to the fastener thereby applying the fastener to the tissue of the patient.

Other aspects of the present invention will be in part apparent and in part pointed out hereinafter.

### BRIEF DESCRIPTION OF THE DRAWINGS

FIG. 1 is a side elevation of a surgical instrument of a first embodiment of the present invention;

FIG. 2 is a side elevation in partial section of a working end portion of the surgical instrument;

FIG. 3 is a cross-sectional view of the instrument taken along line 3-3 of FIG. 2;

FIG. 4 is a cross-sectional view of the instrument taken along line 4-4 of FIG. 2;

FIG. 5 is a cross-sectional view of the instrument taken along line 5-5 of FIG. 2;

FIG. 6 is a side elevation in partial section similar to FIG. 2 but with a retractor element extended;

FIG. 7 is a side elevation in partial section similar to FIG. 2 but with the retractor element engaged;

FIG. 8 is a side elevation in partial section similar to FIG. 2 but with a retractor element retracting;

FIG. 9 is a partial side elevation showing one pair of fasteners applied;

FIG. 10 is a schematic side elevation of a fastener of a second embodiment;

FIG. 11 is a schematic side elevation of a fastener of a third embodiment; and

FIG. 12 is a schematic side elevation of a fastener of a fourth embodiment.

Corresponding reference characters indicate corresponding parts throughout the several views of the drawings.

### DETAILED DESCRIPTION

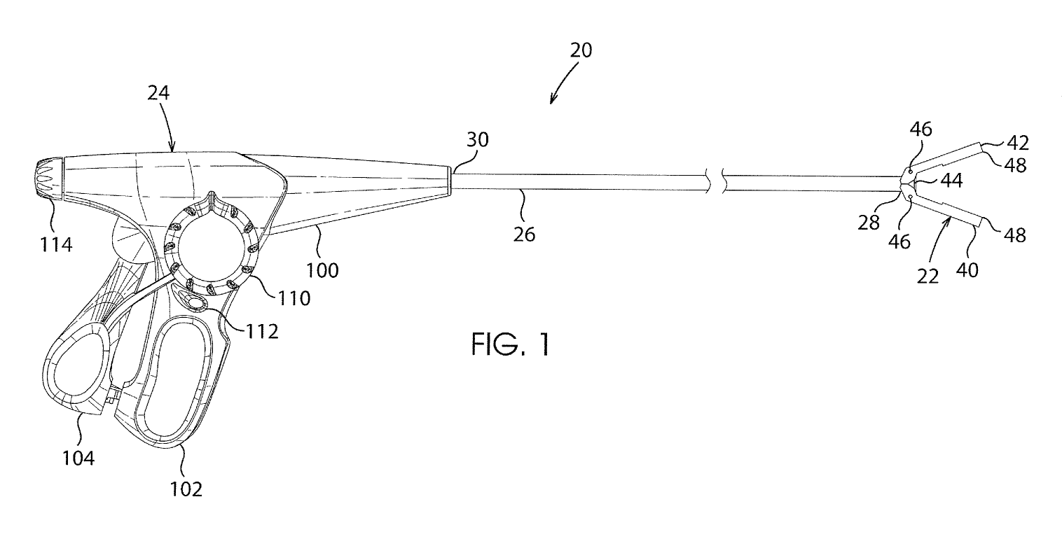

Referring now to the drawings and in particular FIG. 1, a surgical instrument of the present invention is generally designated by the reference number 20. The instrument is specifically adapted for performing endoscopic surgery to fold and fasten tissue. The instrument 20 generally includes an end effector (generally designated by 22) and a handle assembly (generally designated by 24) mounted on opposite ends of an elongate shaft 26. The shaft 26 has a working end 28, on which the end effector 22 is mounted, and a grip end 30, on which the handle assembly 24 is mounted.

In one embodiment, the shaft 26 is a flexible hollow tube having a circular cross section, a length of between about fifty centimeters (cm) and about 150 cm, and an outside diameter of between about 2.5 millimeters (mm) and about five mm. Although the shaft 26 may be made of other materials without departing from the scope of the present invention, in one embodiment the shaft is made of coiled stainless steel wire. In an alternative embodiment, it is envisioned that the shaft 26 may be rigid for laparoscopic use.

As further shown in FIG. 1, the end effector 22 includes opposing first and second jaws 40, 42, respectively, pivotally connected to a connector sleeve 44 mounted on the working end 28 of the shaft 26. A pin 46 holds each jaw 40, 42 on the sleeve 44 so the jaws can pivot between an open position as shown in FIG. 1 and a collapsed position (not shown) in which tips 48 of the jaws touch to reduce an effective cross section of the end effector 22. When applying fasteners, the jaws 40, 42 of the end effector 22 remain in the open position as shown. Thus, when applying fasteners, the jaws 40, 42 of this first embodiment are opposing stationary jaws defining a gap 50. However, when the end effector 22 is not applying fasteners, the jaws 40, 42 may be moved to their collapsed position to enable the end effector to be more easily inserted into and removed from the cavity of the patient. Thus, the jaws 40, 42 of the end effector 22 are selectively moveable to position the jaws in the collapsed configuration in which the gap 50 defined by the jaws is minimized. Although the gap 50 may have other shapes without departing from the scope of the present invention, in one embodiment the gap is generally V-shaped and the jaws 40, 42 are spaced by an angle of between about ten degrees and about thirty degrees. Although the gap 50 may be of other sizes without departing from the scope of the present invention, in one embodiment the gap has a depth of between about fifteen mm and about 35 mm. Other gap shapes and sizes are also contemplated so long as the gap 50 is capable of receiving tissue.

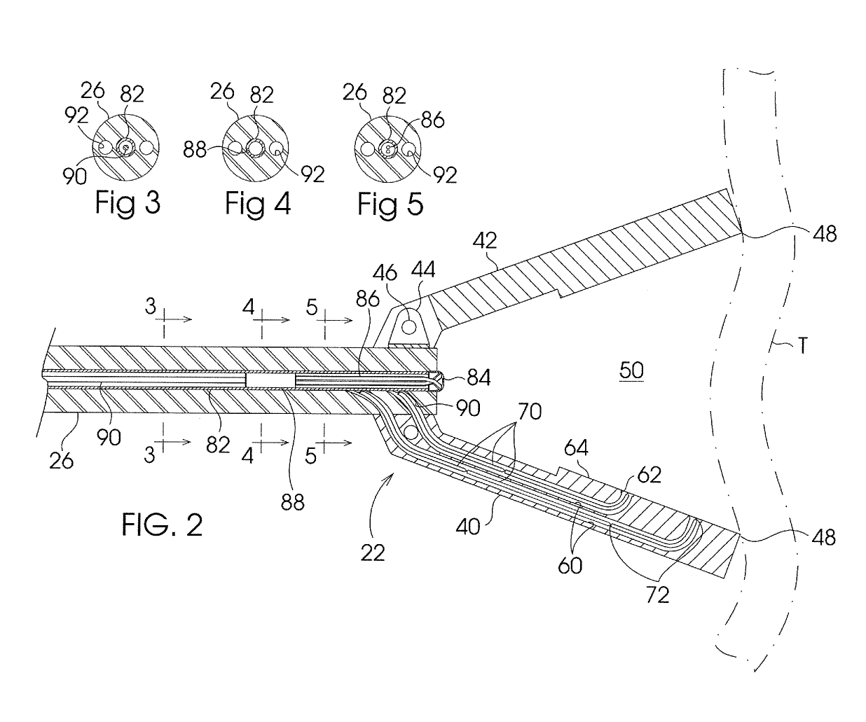

As illustrated in FIG. 2, the first jaw 40 of one embodiment of the end effector 22 includes internal channels 60, each of which end in an opening 62 in a face 64 of the jaw adjacent the gap 50. A series of elongate fasteners 70 are slidably received in the channels. Each of the fasteners 70 includes a pointed tip 72 for reducing trauma to tissue T as the fasteners enter the tissue. Although the fasteners 70 may have other dimensions without departing from the scope of the present invention, in one embodiment each of the fasteners has a length of between about fifteen mm and about 35 mm, and a width of between about one mm and about 1.5 mm. Although the fasteners 70 may be made of other materials without departing from the scope of the present invention, in one embodiment the fasteners 70 are made of Nitinol and have shape memory so that once they are ejected from the channel 60, they assume a desired shape. For example, in some embodiments the fasteners 70 assume curved shapes after they are ejected from the channels 60. In some of these embodiments, the curved shape of the fastener 70 is generally arcuate or circular. In one embodiment, each fastener 70 assumes a generally circular shape once they return to their undeflected shape. Although fasteners 70 of this embodiment may assume circular shapes having other diameters, in one embodiment the fasteners assume a circular shape having an outer diameter of between about five mm and about ten mm. In one embodiment, the fasteners 70 are formed from cylindrical wire.

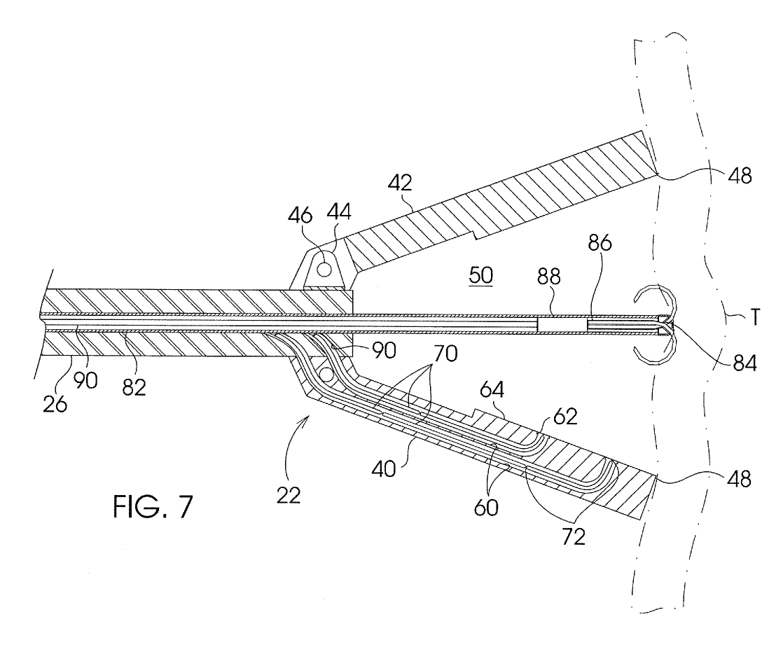

As illustrated in FIGS. 2-5, a tissue retractor, generally designated by 80, is provided inside the shaft 26. The retractor 80 includes a hollow tube 82 slidably mounted in the shaft 26. A cap 84 is mounted on one end of the tube 82 for guiding needles 86 of the retractor 80 as will be described in more detail below. The needles 86 are mounted on a slide 88 that is slideably received in the hollow tube 82. A push rod 90 is mounted on the slide 88 at an end opposite the needles 86. Although the needles 86 may be made of other materials without departing from the scope of the present invention, in one embodiment the needles are made of Nitinol wire having a curved shape when undeflected as shown in FIG. 7. In one particular embodiment, the needles 86 are about 0.5 mm in diameter and form a circular shape when extended from the retractor 80. Although the circular shape of the needles 86 may have other radii of curvature without departing from the scope of the present invention, in one embodiment the needles have a radius of curvature of about fifteen mm. As other features of the tissue retractor mechanism are conventional, they will not be described in further detail. The shaft 26 also includes passages 92 that are aligned with the channels 60 of the jaw 40 for receiving additional fasteners 70.

The handle assembly 24 includes a housing 100 having a scissor grip 102. A thumb lever 104 is pivotally connected to the housing 100 adjacent the scissor grip 102. As the thumb lever 104 is pivoted back and forth relative to the scissor grip 102, a ratchet assembly (not shown) in the housing 100 drives ejector rods (not shown) through the passages 92 in the shaft 26 to sequentially apply individual fasteners 70 to tissue T positioned in the gap 50 between the jaws 40, 42 of the end effector 22. A rotatable wheel 110 is mounted on a side of the housing 100 for selectively driving the retractor tube 82 in and out of the shaft 26 and toward and away from the tips 48 of the jaws 40, 42. Although the rotatable wheel 110 may be operatively connected to the retractor tube 82 by other conventional mechanisms without departing from the scope of the present invention, in one embodiment the wheel is connected to the retractor tube by way of a rack and pinion mechanism (not shown) inside the housing 100. The ratchet may include a biased pawl (not shown) to maintain the retractor tube 82 in a retracted position under the tension from tissue T being retracted. A lever 112 adjacent the wheel 110 is connected to the pawl to disengage the pawl from dogs on the pinion to permit the tube to be extended. A knob 114 is provided on the housing 100 for selectively extending and retracting the needles 86 from the cap 84 of the retractor 80. Although the knob 114 may be operatively connected to the push rod 90 (and thus the needles 86) in other ways without departing from the scope of the present invention, in one embodiment the knob is connected to the push rod by a conventional jack screw mechanism (not shown) mounted in the housing 100.

Although the handle assembly 24 may be made of other materials without departing from the scope of the present invention, in one embodiment the components of the handle assembly are molded from polycarbonate. Although the end effector 22 may be made of other materials without departing from the scope of the present invention, in one embodiment the components of the end effector are made from stainless steel.

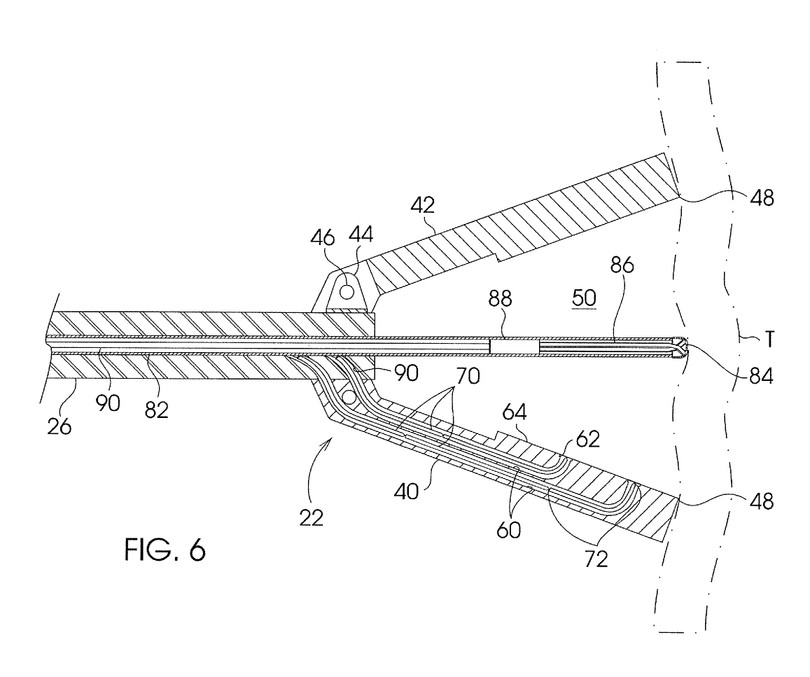

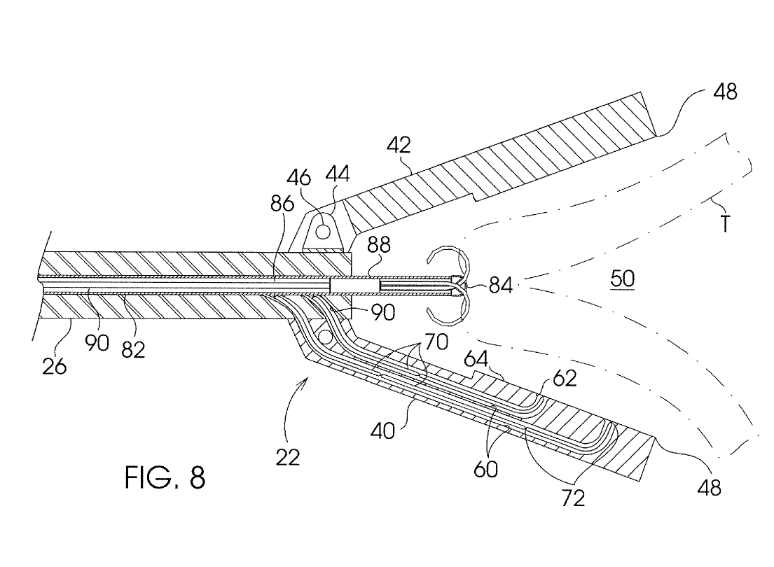

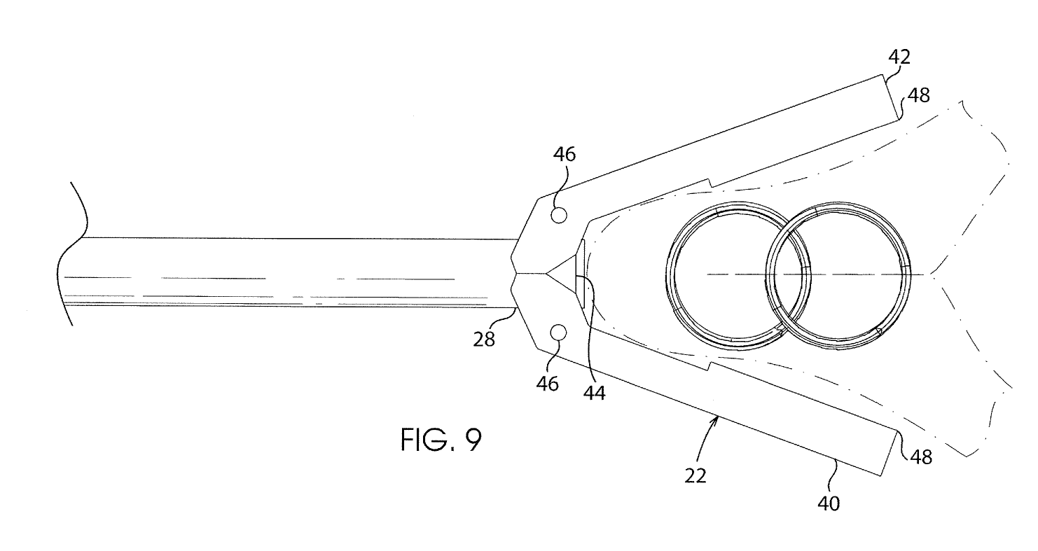

To use the instrument 20 of the present invention, the end effector 22 of the instrument is inserted into a cavity of a patient so that the tips 48 of the jaws 40, 42 are positioned adjacent the tissue T to be fastened as illustrated in FIG. 2. Once in position, the lever 112 is turned to disengage the pawl and the wheel 110 is rotated to extend the retractor tube 82 until the cap 84 of the retractor 80 is at a position beyond the tips 48 of the jaws 40, 42 as illustrated in FIG. 6. In an alternate embodiment of the present method, the retractor tube 82 is extended to a position in which the cap 84 is between the jaws 40, 42. Once the tube 82 is extended to the desired position, the knob 114 is turned to extend the needles 86 from the end of the cap 84 and into the tissue. The needles 86 curve in opposite arcs as the extend as shown in FIG. 7 to effectively connect the retractor 80 to the tissue T. After the retractor 80 is connected to the tissue T, the wheel 110 is turned in an opposite direction to pull the tissue into the gap 50 between the jaws 40, 42 as shown in FIG. 8. Once the tissue T is in a desired position between the jaws 40, 42, the thumb lever 104 is actuated to drive a pair of fasteners 70 into the tissue to maintain the tissue in a folded position as shown in FIG. 9.

As will be appreciated by those skilled in the art, the instrument 20 of the present invention may be manipulated until the tissue T is precisely in a desired location before the fasteners 70 are applied. Moreover, although a jaw 40 having two channels 60 is disclosed in the embodiment described above, a jaw having fewer or more channels for simultaneously applying different numbers of fasteners 70 is also envisioned as being within the scope of the present invention. Still further, although the fasteners 70 are only applied from one jaw 40 in the embodiment described above, it is envisioned that the instrument 20 may be easily modified by those skilled in the art to simultaneously or selectively apply fasteners from both or either jaw 40, 42 without departing from the scope of the present invention. The fasteners 70 are sequentially positioned in the instrument 20 so they can be applied at several positions in the tissue T without removing the end effector from the patient cavity. In an alternative embodiment, only one fastener 70 is loaded in each channel 60. In this alternative embodiment, the instrument 20 is a single shot device.

Many conventional fastener systems have jaws that move relative to the handle assembly 24, making it difficult to precisely position the fastener 40 on the tissue. The instrument 20 of the present invention overcomes this problem by allowing only one jaw to move and keeping the other jaw stationary relative to the handle assembly 24.

FIG. 10 illustrates a second embodiment of a fastener, generally designated by 200, of the present invention. It is envisioned that the fastener 200 could be applied to tissue using an instrument (not shown) having stationary jaws. The fastener 200 of the second embodiment includes a male portion 202 having a detent 204 at one end, and a female portion 206 having a receptacle 208 at one end. The receptacle 208 is sized and positioned on the female portion 206 to receive the detent 204 to connect the respective ends of the male and female portions. The male and female portions, 202, 206, respectively, are joined by a plastic hinge feature 209 permitting the portions to pivot relative to each other.

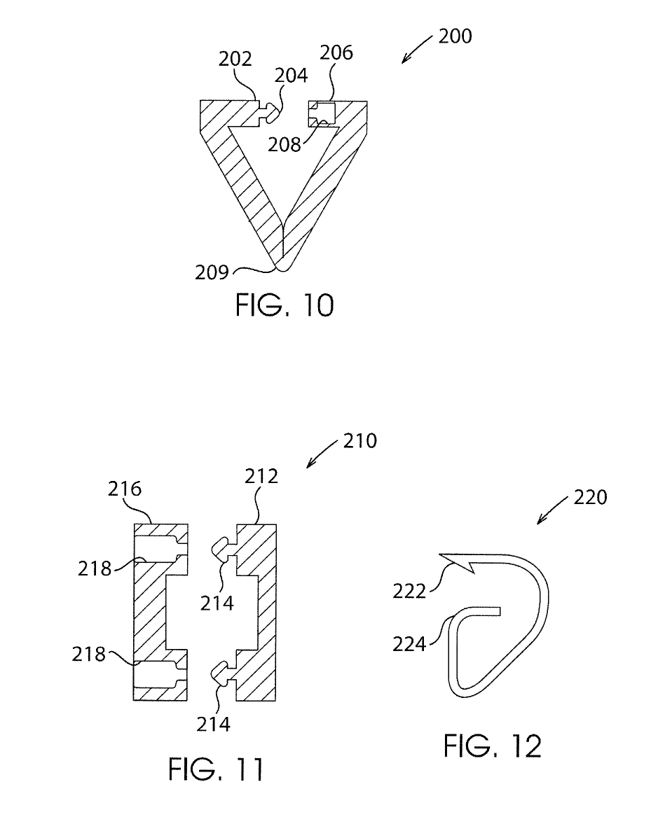

FIG. 11 illustrates a third embodiment of a fastener, generally designated by 210. It is envisioned that the fastener 210 could also be applied to tissue using an instrument (not shown) having stationary jaws. The fastener 210 of the third embodiment includes a male portion 212 having a detent 214 at each end, and a female portion 216 having a receptacle 218 at each end. The receptacles 218 are sized and positioned on the female portion 216 to receive the detents 214 to connect the respective ends of the male and female portions.

FIG. 12 illustrates a fourth embodiment of a fastener, generally designated by 220. It is envisioned that the fastener 220 could also be applied to tissue using an instrument (not shown) similar to the instrument 20 described above. The fastener 220 of the third embodiment includes a pointed barb 222 at one end for advancing the fastener into tissue. The barb 222 discourages the fastener from backing out of the tissue once in position. The fastener 220 also includes a bent or hooked tail 224 that discourages further advancement of the fastener once it is in position. It is envisioned that the hooked tail 224 could be used to pull tissue into a gap between the instrument jaws instead of or in addition to the retractor 80 described above.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions without departing from the scope of the invention, it is intended that all matter contained in the above description or shown in the accompanying drawings shall be interpreted as illustrative and not in a limiting sense.

What is claimed is:

1. A surgical instrument for applying a fastener to tissue of a patient, said instrument comprising:
an elongate shaft having a working end and a grip end opposite said working end;
opposing stationary jaws mounted on the working end of the elongate shaft defining a gap therebetween, said gap being sized and shaped for receiving tissue therein, at least one of said jaws including a fastener ejector directed toward the gap for introducing a fastener into tissue received in the gap to selectively fasten the tissue with the fastener; and
a mechanism operatively connected to the fastener ejector for introducing the fastener into the tissue thereby applying the fastener to the tissue of the patient.

2. A surgical instrument as set forth in claim 1 wherein at least one of the jaws is selectively moveable to position the jaws in a collapsed configuration in which the gap defined by the jaws is minimized.

3. A surgical instrument as set forth in claim 1 further comprising a tissue retractor selectively extendable beyond the jaws and retractable into the gap to position tissue between the jaws for fastening.

4. A surgical instrument as set forth in claim 1 wherein the fastener ejector feeds the fastener along a direction extending generally parallel to the fastener.

5. A surgical instrument as set forth in claim 4 wherein the fastener has a curved shape in an undeflected state and the direction along which the ejector feeds the fastener is tangential to the fastener.

6. A surgical instrument as set forth in claim 5 wherein the fastener has a generally circular shape in the undeflected state.

7. A surgical instrument as set forth in claim 4 wherein the fastener comprises a wire having a point on one end.

8. A surgical instrument as set forth in claim 1 wherein the shaft is flexible.

9. A surgical instrument for applying a fastener to tissue of a patient, said instrument comprising:
an elongate shaft having a working end and a grip end opposite said working end;
opposing jaws mounted on the working end of the elongate shaft defining a gap therebetween, said gap being sized and shaped for receiving tissue therein, at least one of said jaws including an channel ending in an opening adjacent the gap for introducing an elongate fastener into tissue received in the gap to selectively fasten the tissue with the fastener; and
a mechanism operatively connected to the channel for introducing the fastener into the tissue longitudinally with respect to the fastener thereby applying the fastener to the tissue of the patient.

10. A surgical instrument as set forth in claim 9 wherein at least one of the jaws is selectively moveable to position the jaws in a collapsed configuration in which the gap defined by the jaws is minimized.

11. A surgical instrument as set forth in claim 9 further comprising a tissue retractor selectively extendable beyond the jaws and retractable into the gap to position tissue between the jaws for fastening.

12. A surgical instrument as set forth in claim 9 wherein the fastener has a curved shape in an undeflected state and the direction along which the mechanism feeds the fastener is tangential to the fastener.

13. A surgical instrument as set forth in claim 12 wherein the fastener has a generally circular shape in the undeflected state.

14. A surgical instrument as set forth in claim 9 wherein the fastener comprises a wire having a point on one end.

15. A surgical instrument as set forth in claim 9 wherein the shaft is flexible.
